(19) United States
(12) Patent Application Publication
(10) Pub. No.: US 2013/0225501 A1
(43) Pub. Date: Aug. 29, 2013

(54) SKIN COLLAGEN PRODUCTION-PROMOTING AGENT

(71) Applicants: Ken Kato, Hokkaido (JP); Aiko Ono, Hokkaido (JP); Hiroshi Ueno, Hokkaido (JP); Noriko Ueda, Hokkaido (JP); Hiroaki Matsuyama, Hokkaido (JP)

(72) Inventors: Ken Kato, Hokkaido (JP); Aiko Ono, Hokkaido (JP); Hiroshi Ueno, Hokkaido (JP); Noriko Ueda, Hokkaido (JP); Hiroaki Matsuyama, Hokkaido (JP)

(73) Assignee: MEGMILK SNOW BRAND CO., LTD., Hokkaido (JP)

(21) Appl. No.: 13/876,884

(22) PCT Filed: Sep. 29, 2011
(86) PCT No.: PCT/JP2011/072363

§ 371 Date: May 9, 2013

(30) Foreign Application Priority Data
Sep. 30, 2010 (JP) 2010-222101

(51) Int. Cl. A61K 38/57

(52) U.S. Cl. CPC A61K 38/57
USPC 514/18.6; 530/350

(57) ABSTRACT

Skin quality is improved by orally administering a cystatin and/or a degraded product of cystatin in an amount of 10 μg/day or more per adult, or applying a composition that includes a cystatin and/or a degraded product of cystatin in an amount of 0.001 to 2 wt % based on a total amount of the composition.

### TECHNICAL FIELD

The invention relates to a skin collagen production-promoting agent, a food or beverage product for promoting skin collagen production, and a cosmetic for promoting skin collagen production, which are useful in preventing rough skin, skin wrinkles, the loss of elasticity in the skin, or the like. More specifically, the invention relates to a skin collagen production-promoting agent that includes a cystatin and/or a degraded product thereof obtained by degrading a cystatin using a protease as an active ingredient.

### BACKGROUND ART

In recent years, studies on the mechanism of skin have been carried out, and as the results, it has been confirmed that macroscopic causes of dry feeling of the skin and rough skin are complexly involved in the effects of sunlight (ultraviolet), drying, oxidation, and the like in addition to effects due to decrement of the metabolism with aging. It has been found that these effects caused by such factors significantly decrease the amount of collagen that are the main matrix component in the dermis. When a mechanism to keep tension or elasticity of the skin that are maintained by the collagen is destroyed by the effects of ultraviolet or the like, wrinkles or slacks of the skin increase. The collagen molecules can maintain water, so that it helps maintaining skin moisture. Therefore, the skin becomes dry and rough when collagen is destroyed by the external factors. From the above, a skin collagen production-promoting agent that is safe, and can prevent wrinkles and slacks of the skin by promoting biosynthesis of collagen that is one of the main components of the dermis has been desired.

A cystatin is a cysteine protease inhibitor that has an SH group at the active center and inhibits the proteolytic activity of a cysteine protease, and the cystatin has been found in animal tissue, cells, blood, and urine. It has been confirmed that a cystatin has an effect for inhibiting virus growth as a useful effect (see Non-patent Document 1). A composition that contains an amino acid, such as glycine, proline or the like, that increases the amount of protease inhibitor (e.g., cystatin) in skin, and improves the skin water retention rate has been known (see Patent Document 1). However, it has never been known that a cystatin and a degraded product thereof have an effect promoting skin collagen production, and are useful as a skin collagen production-promoting agent.

### PRIOR ART DOCUMENT

### Patent Document

Patent Document 1: JP-A-2008-174522

### Non-Patent Document

Non-patent Document 1: Biochem. Biophys. Res. Commun., vol. 127, p. 1072, 1985

### SUMMARY OF THE INVENTION

### Problems to be Solved by the Invention

An object of the invention is to provide a safe skin collagen production-promoting agent having no problem on safety. Another object of the invention is to provide a food or beverage product promoting skin collagen production, or cosmetic for promoting skin collagen production that includes the skin collagen production-promoting agent.

### Means for Solving the Problems

In order to achieve the above objects, the inventors of the invention have conducted extensive studies on a promoting effect on skin collagen production by using substances that is widely contained in food materials. As a result, the inventors have found that a cystatin or a degraded product thereof obtained by subjecting a cystatin to degradation increases the amount of skin collagen. This finding has led to the completion of the invention.

Specifically, the invention includes the followings:

(1) A skin collagen production-promoting agent including a cystatin and/or a degraded product of cystatin as an active ingredient.

(2) The skin collagen production-promoting agent according to (1), wherein the degraded product is obtained by subjecting a cystatin to degradation using a protease.

(3) The skin collagen production-promoting agent according to (2), wherein the protease is one or more proteases selected from trypsin, pancreatin, chymotrypsin, pepsin, papain, kallikrein, cathepsin, thermolysin, and V8 protease.

(4) The skin collagen production-promoting agent according to any one of (1) to (3), wherein the degraded product has a molecular weight of 500 to 8000.

(5) A food or beverage product for promoting skin collagen production including the cystatin and/or the degraded product of cystatin according to any one of (1) to (4).

(6) A cosmetic for promoting skin collagen production including the cystatin and/or the degraded product of cystatin according to any one of (1) to (4).

(7) A method for improving skin quality including orally administering or applying a cystatin and/or a degraded product of cystatin.

(8) A method for improving skin quality including orally administering a cystatin and/or a degraded product of cystatin in an amount of 10 μg/day or more per adult, or applying a composition that includes a cystatin and/or a degraded product of cystatin in an amount of 0.001 to 2 wt % based on a total amount of the composition.

(9) The method according to (7) or (8), wherein the degraded product of cystatin is obtained by subjecting a cystatin to degradation using a protease.

(10) The method according to (9), wherein the protease is one or more proteases selected from trypsin, pancreatin, chymotrypsin, pepsin, papain, kallikrein, cathepsin, thermolysin, and V8 protease.

(11) The method according to (9), wherein the degraded product of cystatin has a molecular weight of 500 to 8000.

### Effects of the Invention

According to the invention, there are provided a skin collagen production-promoting agent, a food or beverage product for promoting skin collagen production, and a cosmetic for promoting skin collagen production that each includes a cystatin and/or a degraded product thereof as an active ingredient. The skin collagen production-promoting agent, the food or beverage product for promoting skin collagen production, and the cosmetic for promoting skin collagen production exhibit a promoting effect on skin collagen production, and thus may be useful for preventing or treating skin wrinkles, the loss of elasticity in the skin, skin dry feeling, and rough skin.

### EMBODIMENTS FOR CARRYING OUT THE INVENTION

A skin collagen production-promoting agent according to the invention includes a cystatin and/or a degraded product thereof that is obtained by subjecting a cystatin to degradation using a protease as an active ingredient.

Even if the cystatin is derived from any source, it may be used for the skin collagen production-promoting agent according to the invention. For example, the gene sequences of human cystatins and bovine cystatins have been already determined, and human cystatins and bovine cystatins can be produced by a gene recombination technique. A cystatin produced by a gene engineering technique may also be used for the skin collagen production-promoting agent according to the invention. A relatively large amount of cystatins are contained in bovine colostrum. A cystatin collected from cow milk may also be used for the skin collagen production-promoting agent according to the invention. A cystatin can also be collected from a cell culture solution. Such a cell-derived cystatin may also be used in the invention.

For example, a milk-derived cystatin can be produced by a known method (see JP-A-2000-281587, etc). Specifically, a cystatin can be obtained from raw milk, milk powder, skim milk, recombined milk, or the like through a heat treatment, a salting treatment, an ethanol treatment, a chromatographic treatment, such as ion-exchange chromatography or gel filtration chromatography, an ultrafiltration treatment, and the like.

As the degraded product of cystatin, peptide mixture obtained by subjecting a cystatin to limited degradation using a protease, such as trypsin, pancreatin, chymotrypsin, pepsin, papain, kallikrein, cathepsin, thermolysin, V8 protease or the like so that the cystatin has a molecular weight of 8000 or less may be used. Note that the lower limit of the molecular weight is preferably 500 or more.

The skin collagen production-promoting agent according to the invention exerts a promoting effect on skin collagen production through oral administration or application. In the case of oral administration of the skin collagen production-promoting agent of the present invention, the cystatin or the degraded product thereof (i.e., active ingredient) may be orally administered as it is (without any additional treatment). Of course, the cystatin or the degradation product may be used after it is formulated into a powder, granule, tablet, capsule, drink, or the like according to the conventional method. In the present invention, an oral preparation such as a powder, granule, tablet, or capsule is formulated by using a vehicle such as starch, lactose, sucrose, mannitol, carboxymethylcellulose, cornstarch, or inorganic salts, or the like according to the conventional method. For this kind of formulation, there may be used, in addition to the above described vehicles, a binder, disintegrator, surfactant, lubricant, fluidity promoter, colorant, flavor, or, the like. More specifically, examples of the binder include starch, dextrin, powdered acacia, gelatin, hydroxypropyl starch, sodium carboxymethylcellulose, methyl cellulose, crystalline cellulose, ethyl cellulose, polyvinylpyrrolidone, for example. Examples of the disintegrator include starch, hydroxypropyl starch, carboxymethylcellulose, sodium carboxymethylcellulose, crosslinked sodium carboxymethylcellulose, crystalline cellulose, and the like. Examples of the surfactant include soybean lecithin, sucrose fatty acid ester, and the like. Examples of the lubricant include talc, wax, sucrose fatty acid ester, hydrogenated vegetable oils, and the like. Examples of the fluidity promoter include silicic anhydride, dried aluminum hydroxide, magnesium silicate, and the like.

The cystatin, the degraded product thereof, or a preparation prepared using the cystatin or the degraded product may be incorporated in a nutrient supplement, food or beverage product, or the like without any additional treatment or after being formulated. In addition, it is expected that the promoting effect on skin collagen production is further improved when the cystatin or the degraded product is incorporated in combination with an ingredient such as vitamin C that is generally considered to be effective for collagen production. Since the cystatin or the degraded product thereof is relatively stable against heat, a raw material including the cystatin or the degraded product thereof can be heat-sterilized under a general condition.

When applying the skin collagen production-promoting agent according to the invention, the skin collagen production-promoting agent may be incorporated in a known ingredient that is generally used depending on the intended use to thereby prepare various dosage forms such as a liquid formulation, a solid formulation, a semi-solid formulation, or the like. Examples of a preferable composition include an ointment, gel, cream, spray, patch, lotion, powder, and the like. For example, the skin collagen production-promoting agent according to the invention may be mixed with a hydrocarbon such as petroleum, higher fatty acid lower alkyl ester such as stearyl alcohol or isopropyl myristate, animal oil and fat such as lanoline, a polyhydric alcohol such as glycerin, surfactant such as glycerin fatty acid ester, or polyethyleneglycol monostearate, inorganic salt, wax, resin, water, and if necessary, preservative such as methyl parahydroxybenzoate or butyl parahydroxybenzoate, to thereby produce a cosmetic or pharmaceutical product for promoting skin collagen production.

The effective amount of the skin collagen production-promoting agent according to the invention is appropriately determined depending on the dosage form, the administration method, the intended use, and the age, the weight, and the symptom of a patient to whom the skin collagen production-promoting agent is administered, and it is not constant. Note that the results of animal experiments using rats revealed that it is necessary to administer the cystatin and/or the degraded product thereof in an amount of 10 μg or more per 1 kg of rat weight in order to exert the promoting effect on skin collagen production. Therefore, according to the extrapolation method, when the cystatin and/or the degraded product thereof are taken in an amount of 10 μg/day or more per adult, the effect may be expected. Accordingly, the cystatin and/or the degraded product thereof may be incorporated in a food or beverage product, or may be administered as a drug so that the above amount is ensured. Note that the cystatin and/or the degraded product thereof may optionally be administered several times in a day.

The effective amount of the skin collagen production-promoting agent according to the invention for application varies depending on the dosage form. The cystatin and/or the degraded product thereof may preferably be incorporated in the amount of 0.001 to 2 wt % on the basis of the amount of all compositions to be applied. Note that the amount of the cystatin and/or the degraded product thereof may be increased when the composition is diluted during use as a bath additive.

### EXAMPLES

The invention is further described below in detail by way of examples, and test examples. Note that the following examples merely illustrate some embodiments of the invention, and should not be construed as limiting the invention.

### Example 1

A column charged with 3,000 g of S-sepharose was sufficiently washed with deionized water. After passing 10,000 1 (liter) of skim milk through the column, the column was sufficiently washed with deionized water, and the mixture was then subjected to elution with a linear gradient of 0.1 to 1.0 M sodium chloride. The resulting fraction was heated at 90° C. for 10 minutes, and subjected to centrifugation to remove precipitates. The eluted fraction containing a cow milk-derived basic cystatin was fractionated again by Mono S ion-exchange chromatography. Next, the fraction was subjected to Mono Q ion-exchange chromatography and Superose 12 gel filtration chromatography using an FPLC system, and then subjected to hydroxyapatite chromatography and C4 reversed-phase chromatography sequentially using an HPLC system to obtain 58 mg of a cystatin (fraction A). The cystatin thus obtained may be used as the skin collagen production-promoting agent without any additional treatment.

### Example 2

10,000 1 of a 5% milk serum protein solution was heated at 90° C. for 10 minutes, and subjected to centrifugation to remove precipitates. A column was charged with a carrier prepared by binding carboxymethylated papain to Tresyl-Toyopearl (manufactured by Tosoh Corporation). After equilibration with a 0.5M sodium chloride solution, the milk serum protein solution was passed through the column. The column was then sequentially washed with a 0.5 M sodium chloride solution and a 0.5 M sodium chloride solution containing 0.1% Tween 20. Next, a cysteine protease was eluted with a 20 mM acetic acid-0.5 M sodium chloride solution. The eluted fraction was immediately neutralized with 1 M sodium hydroxide solution, subjected to Mono S anion-exchange chromatography, and then subjected to hydroxyapatite chromatography and C4 reversed-phase chromatography using an HPLC system to obtain 48 mg of a cow milk-derived basic cystatin (fraction B). The cystatin thus obtained may be used as the skin collagen production-promoting agent without any additional treatment.

### Example 3

25 mg of the fraction A obtained in Example 1 was suspended in 100 ml of water. After the addition of pancreatin so that the final concentration thereof was 1%, the mixture was subjected to an enzymatic treatment at 37° C. for 5 hours. After inactivating the protease by heat-treating the mixture at 90° C. for 5 minutes, the mixture was freeze-dried to obtain 23 mg of a degraded product of cystatin (fraction C). 25 mg of the fraction B obtained in Example 2 was treated in the same manner as described above to obtain 24 mg of a degraded product of cystatin (fraction D). The degraded products of cystatin thus obtained had a molecular weight of 8000 or less, and may be used as the skin collagen production-promoting agent without any additional treatment.

### Test Example 1

The promoting effect on skin collagen production of the fraction A obtained in Example 1 and the fraction C obtained in Example 3 was determined by animal experiments using rats. 7-week-old Wistar male rats were divided into following five groups (n=6); a group to which physiological saline was administered (group A), a group to which the fraction A obtained in Example 1 was administered in an amount of 10 μg per 1 kg of rat weight (group B), a group to which the fraction A obtained in Example 1 was administered in an amount of 100 μg per 1 kg of rat weight (group C), a group to which the fraction C obtained in Example 3 was administered in an amount of 10 μg per 1 kg of rat weight (group D), and a group to which the fraction C obtained in Example 3 was administered in an amount of 100 μg per 1 kg of rat weight (group E). Every rat was kept for 10 weeks during which physiological saline or the fraction was administered using a sonde once a day. The amount of collagen in the skin was determined by treating the dermis of each rat in accordance with the method by Nimni et al. (see Arch. Biochem. Biophys., p. 292, 1967), and measuring the hydroxyproline content in the soluble fraction. Hydroxyproline is a special kind of amino acid contained only in collagen, and accounts for about 10% of all amino acids that constitute collagen. Therefore, the amount of collagen can be estimated (see Asano et al., BioIndustry, p. 12, 2001). The results are shown in Table 1.

TABLE 1
Hydroxyproline content (μg/ml)

| | |
|---|---|
| Group A | 0.3 ± 0.1 |
| Group B | 0.9 ± 0.2* |
| Group C | 1.3 ± 0.3* |
| Group D | 0.8 ± 0.1* |
| Group E | 1.4 ± 0.2* |

The value shown in Table 1 indicates “mean±standard deviation” (n=6). The symbol “*” indicates that there was a significant difference in comparison with the group A that is a control group (p<0.05).

As shown in Table 1, the amount of hydroxyproline in the soluble fraction after 10 weeks was significantly higher in the groups B, C, D, and E as compared with the group A. It was thus confirmed that the cystatin and the degraded product thereof have a promoting effect on skin collagen production, and are useful as a skin collagen production-promoting agent. It is revealed that the promoting effect on skin collagen production is obtained when the cystatin or the degraded product thereof is administered in an amount of at least 10 μg per 1 kg of rat weight.

### Test Example 2

The promoting effect on skin collagen production of the fraction B obtained in Example 2 and the fraction D obtained in Example 3 was determined by experiments using a normal human fibroblast strain (CCD45SK (ATCCRL 1506) collected from the skin of a white female). The cells were seeded to a 24-well plate at a concentration of 4×10⁴ cells/well/0.4 ml using a modified Eagle medium (MEM) (“10-101” manufactured by Dainippon Pharmaceutical Co., Ltd.) containing 10 vol % bovine fetal serum (hereinafter abbreviated as FBS), and cultured at 37° C. for 24 hours under 5% carbon dioxide and saturated water vapor, and the medium was replaced with an MEM containing 0.6 vol % FBS. Thereafter, the fraction B obtained in Example 2 and the fraction D obtained in Example 3 were added to each well at a concentration of 0.1 vol %, and the cells were cultured for 24 hours. After that, β-aminopropionitrile and tritiated L-proline at concentration of 50 μg/ml and 1 μCi/ml, respectively, and the cells were cultured for additional 24 hours to obtain a culture solution. A collagen fraction was fractionated from the culture solution in accordance with the method of Webster et al. (see Analytical Biochemistry, p. 220, 1979), and the amount of radioactivity that had been taken in the collagen fraction was measured. Note that similar test was performed without adding the cystatin or the degraded product thereof as a control. The results are shown in Table 2.

TABLE 2

| | Collagen production (%) |
|---|---|
| Control | 100 ± 3 |
| Milk protein fraction | 257 ± 10* |
| (Invention product 2) | |
| Degradation product of milk protein fraction | 246 ± 7* |
| (Invention product 4) | |

The value shown in Table 1 indicates “mean±standard deviation” (n=6). The symbol “*” indicates that there was a significant difference in comparison with the group A that is a control group (p<0.05).

As shown in Table 2, the promoting effect on skin collagen production obtained in the groups to which the cystatin or the degraded product thereof was added was equal to or more than twice as compared with that of the group (control) to which the cystatin or the degraded product thereof was not added. The results revealed that the cystatin and the degraded product thereof affect skin fibroblasts, and have a promoting effect on skin collagen production, and are useful as a skin collagen production-promoting agent.

### Example 4

A beverage product for promoting skin collagen production having the composition shown in Table 3 was produced according to the conventional method using the ingredients shown in Table 3. The beverage thus produced had a good flavor, the flavor did not deteriorate even after 1 year storage at room temperature, and there was no problem such as generation of precipitates and the like.

TABLE 3

| | |
|---|---|
| Mixed isomerized sugar | 15.0 (wt %) |
| Fruit juice | 10.0 |
| Citric acid | 0.5 |
| Skin collagen production-promoting | 0.1 |
| agent (Invention product 2) | |
| Flavor | 0.1 |
| Mineral mixture | 0.1 |
| Water | Balance |

### Example 5

Dough having the composition shown in Table 4 was prepared according to a conventional method, then formed and baked to produce biscuits for promoting skin collagen production.

TABLE 4

| Flour | 50.0 (wt %) |
|---|---|
| Sugar | 20.0 |
| Salt | 0.5 |
| Margarine | 12.5 |
| Egg | 12.5 |
| Water | 3.5 |
| Mineral mixture | 0.8 |
| Fraction D (Invention product 3) | 0.2 |

### Example 6

A skin collagen production-promoting agent having the composition shown in table 5 was produced according to a conventional method.

TABLE 5

| Hydrous crystalline glucose | 90.5 (wt %) |
|---|---|
| Mineral mixture | 5.0 |
| Fraction A (Invention product 1) | 3.0 |
| Sugar ester | 1.0 |
| Flavor | 0.5 |

### Example 7

A lotion having the composition shown in Table 5 was produced according to the conventional method.

TABLE 6

| | |
|---|---|
| Glycerin | 3.0 (wt %) |
| 1,3-Butylene glycol | 3.0 |
| Polyoxyethylene sorbitan monooleate (20E.0.) | 0.5 |
| Methyl parahydroxybenzoate | 0.15 |
| Citric acid | 0.1 |
| Sodium citrate | 1.0 |
| Flavor | 0.05 |
| Fraction C (Invention product 3) | 0.05 |
| Purified water | Balance |

### Example 8

Cream having the composition shown in Table 7 was produced according to the conventional method.

TABLE 7

| | |
|---|---|
| Liquid paraffin | 5.0 (wt %) |
| White beeswax | 4.0 |
| Cetanol | 3.0 |
| Squalane | 10.0 |
| Lanolin | 2.0 |
| Stearic acid | 1.0 |
| Polyoxyethylene sorbitan monooleate (20E.0.) | 1.5 |
| Glyceryl monostearate | 3.0 |
| 1,3-Butylene glycol | 6.0 |
| Methyl parahydroxybenzoate | 1.5 |
| Flavor | 0.1 |
| Fraction B (Invention product 2) | 0.5 |
| Purified water | Balance |

### Test Example 3

A practical use test was performed using the lotion obtained in Example 7 and the cream obtained in Example 8. As a lotion and cream for comparison, there were used lotion and cream having the same compositions as those in Example 7 or 8, except that the cystatin was not added, was used as a comparative product. Twenty adult females having face with the loss of elasticity in the skin or fine wrinkles and having dry skin were divided at random into two groups each having 10 subjects (groups A and B), and twenty adult females having rough hand skin were divided at random into two groups each having 10 subjects (groups C and D). 2 g of the lotion of the invention, 2 g of the lotion for comparison, 2 g of the cream of invention, and 2 g of the cream for comparison were applied to the faces of group A, the faces of group B, the hands and fingers of group C, and the hands and fingers of group D, respectively, in the same way as usual twice in a day for 10 days. The results are shown in Table 8.

TABLE 8

| | Dry feeling | Rough skin | Wrinkles | The loss of elasticity |
|---|---|---|---|---|
| Group A | ++ | ++ | + | ++ |
| Group B | ± | ± | ± | ± |
| Group C | + | ++ | Not measured | Not measured |
| Group D | ± | ± | Not measured | Not measured |

++: A significant change was observed after 10 days
+: A change was observed after 10 days
±: No change was observed after 10 days

As shown in Table 8, it was revealed that the lotion of the invention significantly improved skin dry feeling, rough skin, and the like, and exhibited an excellent promoting effect on skin collagen production as compared with the comparative lotion. It was also revealed that the cream of the invention significantly improved skin dry feeling, chapping, and the like, and suppressed spontaneous exacerbation, such as rough skin as compared with the comparative cream.

1. A skin collagen production-promoting agent comprising a cystatin and/or a degraded product of cystatin as an active ingredient.

2. The skin collagen production-promoting agent according to claim 1, wherein the degraded product of cystatin is obtained by subjecting a cystatin to degradation using a protease.

3. The skin collagen production-promoting agent according to claim 2, wherein the protease is one or more proteases selected from trypsin, pancreatin, chymotrypsin, pepsin, papain, kallikrein, cathepsin, thermolysin, and V8 protease.

4. The skin collagen production-promoting agent according to claim 1, wherein the degraded product of cystatin has a molecular weight of 500 to 8000.

5. A food or beverage product for promoting skin collagen production comprising the cystatin and/or the degraded product of cystatin according to claim 1.

6. A cosmetic for promoting skin collagen production comprising the cystatin and/or the degraded product of cystatin according to claim 1.

7. A method for improving skin quality comprising orally administering or applying a cystatin and/or a degraded product of cystatin.

8. A method for improving skin quality comprising orally administering a cystatin and/or a degraded product of cystatin in an amount of 10 μg/day or more per adult, or applying a composition that includes a cystatin and/or a degraded product of cystatin in an amount of 0.001 to 2 wt % based on a total amount of the composition.
